(19) United States
(12) Patent Application Publication
(10) Pub. No.: US 2013/0345723 A1
(43) Pub. Date: Dec. 26, 2013

(54) APPARATUS FOR REMOVING DEAD SKIN

(71) Applicant: Yong-Neung Song, Incheon (KR)

(72) Inventor: Yong-Neung Song, Incheon (KR)

(21) Appl. No.: 14/003,817

(22) PCT Filed: Mar. 25, 2011
(86) PCT No.: PCT/KR2011/002063

§ 371 Date: Sep. 9, 2013

(30) Foreign Application Priority Data
Mar. 11, 2011 (KR) 10-2011-0022087

(51) Int. Cl. A61B 17/3205

(52) U.S. Cl. CPC A61B 17/3205
USPC 606/131

(57) ABSTRACT

The present invention relates to an apparatus for removing dead skin, and more particularly, to an apparatus for removing dead skin which can safely remove hardened skin or dead skin generated on the soles, heels, and palms without damaging normal skin, and simultaneously easily remove the dead skin remaining within the apparatus for removing dead skin. To this end, the apparatus for removing dead skin includes a tube-shaped body on one side of which a blade for removing dead skin is disposed, and a grasping member disposed outside the body.

### TECHNICAL FIELD

The present invention relates an apparatus for removing dead skin, and more particularly, to an apparatus for removing dead skin which can safely remove hardened skin or dead skin generated on the soles, heels, and palms without damaging normal skin, and simultaneously easily remove dead skin particles remaining within the apparatus for removing dead skin.

### BACKGROUND ART

Generally speaking, dead skin (so called “keratin”) naturally forms on a skin surface of a human body and combines with sebum and other pollutants for thereby forming plague which blocks hair follicle and damaging a skin health and beauty.

Therefore, it needs to periodically remove dead skin for the sake of a skin beauty and health.

As shown in FIG. 1, a conventional apparatus requires a polishing device with a handle as a polishing means. In another way, as shown in FIG. 2, a conventional apparatus uses a dead skin device formed in a disposal type razor for thereby polishing a dead skin from a skin surface.

The conventional dead skin removing apparatus has features in that the dead skin formed at a wider portion such as soles can be easily eliminated; but the dead skin or hardened skin formed at a narrow portion such as between fingers or toes or formed at a bent portion cannot be easily eliminated.

Dead skin particles producing when dead skin or hardened skin is removed using a conventional dead skin removing apparatus may fly around, thus contaminating surroundings.

### DISCLOSURE OF INVENTION

Accordingly, it is an object of the present invention to provide an apparatus for removing dead skin which overcomes the problems encountered in the conventional art and makes it possible to easily remove dead skin or hardened skin formed at soles, heels or palms or between fingers or toes using a circular dead skin removing blade without damaging skin.

It is another object of the present invention to provide an apparatus for removing dead skin which makes it possible to keep a dead skin removing apparatus clean all the time in such a way to easily remove dead skin particles at the dead skin removing apparatus using a dead skin particle removing means which is configured to remove dead skin or hardened skin remaining in a dead skin removing apparatus after dead skin or hardened skin is removed.

It is further another object of the present invention to provide an apparatus for removing dead skin which has features in that a handle is provided for easily grasping a dead skin removing apparatus, and a protrusion ring surrounding a dead skin removing apparatus is provided. When a dead skin removing apparatus comes into contact with a skin, the protrusion ring also contacts with the skin in order to help the dead skin removing apparatus be at a right angle. With these features, even when a user removes a dead skin by applying a certain over force, the apparatus does not over penetrate deep into the skin, so the skin is not damaged, whereby the dead skin or hardened skin can be removed in safe.

It is still further another object of the present invention to provide an apparatus for removing dead skin which has features in that since the dead skin removing blade is formed in a circular shape, the dead skin can be efficiently removed in all the directions, so the present invention helps easily remove dead skin or hardened skin.

To achieve the above objects, there is provided an apparatus for removing dead skin, comprising a tubular body having a dead skin removing blade at its one side; and a grasping member which is formed at an outer portion of the body.

In another embodiment of the present invention, there is provided an apparatus for removing dead skin, comprising a tubular body having a dead skin removing blade at its one side; a support member formed at an outer side of the body; and a grasping member to which the support member is fixedly engaged.

Here, a protrusion ring is formed concentrically to a dead skin removing blade of the body.

The protrusion ring is formed at a grasping member.

The protrusion ring is formed at a support member.

The protrusion ring has a plurality of cut-away grooves.

At this time, there is further provided a cap which is fixedly engaged to an outer surface of the protrusion ring.

In addition, a polishing member is formed at an outer surface of the grasping member.

A dead skin particle removing part is provided at an inner side of the body.

Here, the dead skin removing part comprise a support ring which is provided at an inner surface of the grasping member; a cover member which serves to cover the reverse direction of the direction where the body is engaged and has a slide hole in a stair-shape at its one side; a button which is engaged to the slide hole of the cover member; a connection rod which is provided at an inner side of the grasping member and one side of which is engaged to the button; a removing member which is fixedly engaged to the other side to which the button is fixedly engaged; and an elastic member which is engaged to the connection rod and is positioned between the support ring and the button.

The dead skin particle removing part comprises a rotational body in which a screw groove is formed at an inner surface of the grasping member, and an insertion groove is formed at an inner surface of the other side to which the body is fixedly engaged, and a handle is inserted into the inner side of the grasping member and has an insertion protrusion engaged to the insertion groove and is stair-shaped and protrudes toward an outer side of the grasping member, and an ascending and descending guide groove with crossing engaging grooves is formed at both ends in a longitudinal direction, and the rotational body has an ascending and descending member inserted into a through hole of the body as one side protrudes outwardly and is formed in a stair-shape, and at a lateral side is provided an ascending and descending holder having a guide protrusion which passes through the ascending and descending guide groove and is engaged to the screw groove of the grasping member.

The dead skin particle removing part comprises a slide rod in which a tubular guide member is provided at an inner side of the grasping member, and a rotational body is rotatable-engaged to the bottom of the grasping member, and a tubular engaging member is formed at one inner side of the rotational body and is positioned on the same axis as the guide member, and a screw groove is formed at an inner surface of the engaging member, and one end portion of the slide rod passes through the guide member and is positioned in the through hole of the body, and the other side is engaged to the engaging member, and a ball is provided at the engaging part of the engaging member.

At this time, the slide rod comprises an engaging protrusion.

In addition, the dead skin removing blade comprises an assistant dead skin removing blade at its outer side.

The bodies are provided in multiple numbers.

### ADVANTAGEOUS EFFECTS

The present invention makes it possible to easily remove dead skin or hardened skin formed at soles, heels or palms or between fingers or toes using a circular dead skin removing blade without damaging skin.

In addition, the present invention makes it possible to keep a dead skin removing apparatus clean all the time in such a way to easily remove dead skin particles at the dead skin removing apparatus using a dead skin particle removing means which is configured to remove dead skin or hardened skin remaining in a dead skin removing apparatus after dead skin or hardened skin is removed.

In addition, in the present invention, a handle is provided for easily grasping a dead skin removing apparatus, and a protrusion ring surrounding a dead skin removing apparatus is provided. When a dead skin removing apparatus comes into contact with a skin, the protrusion ring also contacts with the skin in order to help the dead skin removing apparatus be at a right angle. With these features, even when a user removes a dead skin by applying a certain over force, the apparatus does not over penetrate deep into the skin, so the skin is not damaged, whereby the dead skin or hardened skin can be removed in safe.

In addition, since the dead skin removing blade is formed in a circular shape, the dead skin can be efficiently removed in all the directions, so the present invention helps easily remove dead skin or hardened skin.

### BRIEF DESCRIPTION OF DRAWINGS

FIGS. 1 and 2 are views illustrating a conventional dead skin removing apparatus.

FIG. 3 is a perspective view illustrating a dead skin removing apparatus according to the present invention.

FIG. 4 is a cross sectional view illustrating a dead skin removing apparatus according to the present invention.

FIG. 5 is an enlarged view illustrating a portion “A” of FIG. 4.

FIG. 6 is a plane view illustrating a dead skin removing apparatus according to the present invention.

FIG. 7 is a view illustrating an operational relationship of a dead skin removing apparatus according to the present invention.

FIG. 8 is a view illustrating a dead skin removing apparatus according to another embodiment of the present invention.

FIG. 9A is a view illustrating a dead skin removing apparatus according to further another embodiment of the present invention.

FIG. 9B is a view illustrating an operational relationship of FIG. 9A.

FIGS. 10A and 10B are views illustrating a dead skin removing apparatus according to still further another embodiment of the present invention.

FIG. 10C is a cross sectional view taken along line B-B of FIG. 10B.

FIG. 11 is a view illustrating a dead skin removing apparatus according to still further another embodiment of the present invention.

FIGS. 12A to 12C are views illustrating a dead skin removing apparatus according to still further embodiment of the present invention.

FIG. 13 is a view illustrating a dead skin removing apparatus according to still further another embodiment of the present invention.

FIG. 14A is an enlarged view of a portion “C” of FIG. 13.

FIG. 14B is an enlarged view of a portion “D” of FIG. 13.

FIG. 15 is a view illustrating an operational relationship of FIG. 13.

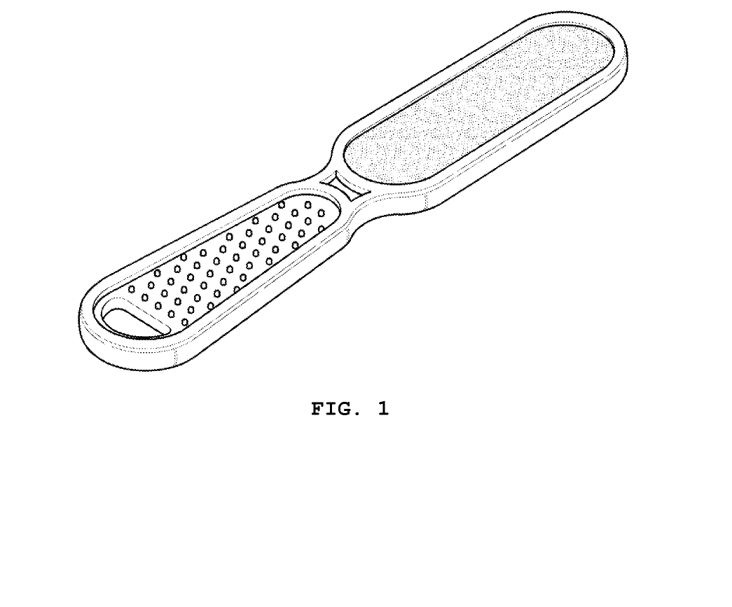

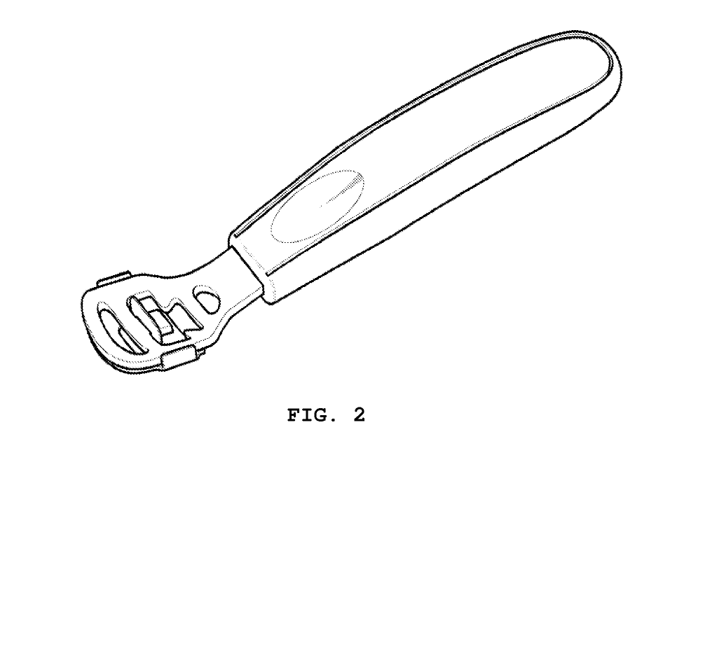

### BEST MODES FOR CARRYING OUT THE INVENTION

The dead skin removing apparatus according to the present invention will be described with reference to the accompanying drawings.

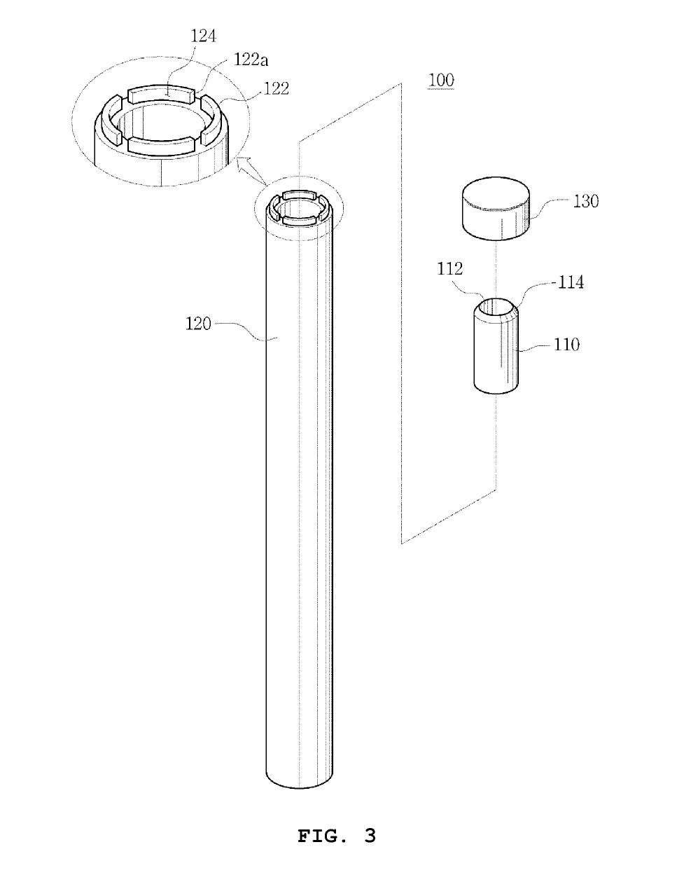

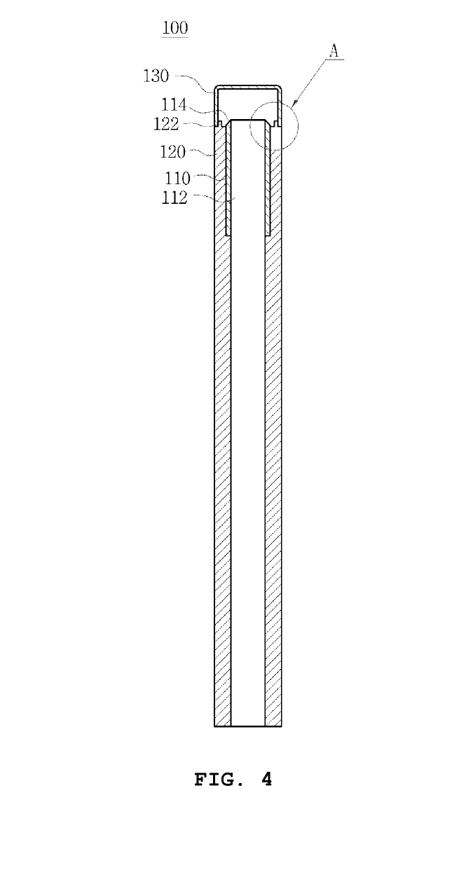

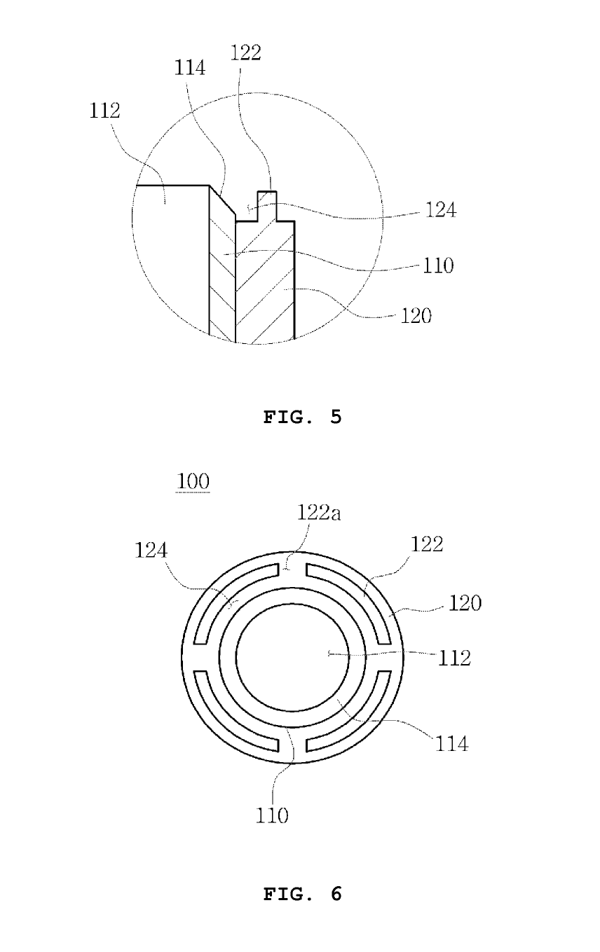

FIG. 3 is a perspective view illustrating a dead skin removing apparatus according to the present invention. FIG. 4 is a cross sectional view illustrating a dead skin removing apparatus according to the present invention. FIG. 5 is an enlarged view illustrating a portion “A” of FIG. 4. FIG. 6 is a plane view illustrating a dead skin removing apparatus according to the present invention.

As shown in FIGS. 3 to 6, the dead skin removing apparatus 100 according to the present invention comprises a body 110 having a through hole 112 formed in its interior and a dead skin removing blade 114 disposed at one side in a longitudinal direction for removing dead skin.

At this time, the body 100 is formed in a tubular shape and has a dead skin removing blade 114 an outer side of which is slanted.

At an outer side of the body 100 is provided a grasping member 120 that a user can easily grasp.

The grasping member 120 is fixedly engaged in such a way that an outer side of the dead skin removing blade 114 of the body 110 is covered, and an end portion of the dead skin removing blade 114 protrudes a certain length and is fixedly engaged to an outer side of the body 110.

The grasping member 120 is formed in a concave groove shape at the portions where the users fingers reach so that the user can tightly grasp.

From one side of the grasping member 120, more specifically, in the direction that the dead skin removing blade 114 of the body 110 is formed, protrudes a protrusion ring 122 which is spaced apart from an outer side of the body 110 for thereby forming a collection chamber 124 between itself and an outer surface of the body 110.

The protrusion ring 122 comprises a plurality of cut-away grooves 122a, and a cap 130 is engaged to the protrusion ring 122 for thereby covering the dead ski removing blade 114 of the body 110.

Meanwhile, as is best seen in the drawings, the body 110 is formed in a short tubular shape and may be so engaged or injection-molded that the protrusion ring 122 of the grasping member 120 can outwardly protrude. As is not shown in the drawings, the body 110 has the same length as the grasping member 120. The body 110 can be so engaged or injection-molded that the protrusion ring 122 formed at the grasping member 120 can outwardly protrude.

The operations of the dead skin removing apparatus according to the present invention will be described.

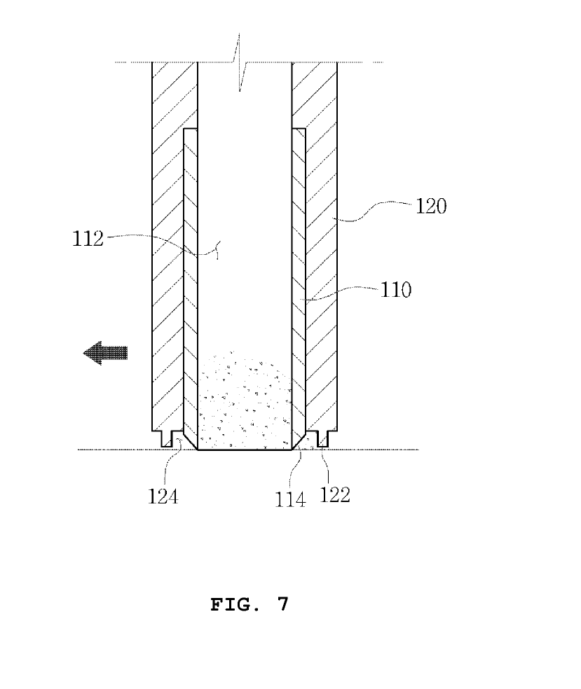

FIG. 7 is a view illustrating an operational relationship of a dead skin removing apparatus according to the present invention.

As best seen therein, when the dead skin removing apparatus 100 according to the present invention is actually used to remove dead skin, corn or hardened skin, the dead skin removing blade 114 is positioned at a dad skin portion after a user grasps the grasping member 120.

In the above state, the use repeatedly rubs holding the dead skin removing apparatus 100 in all directions for thereby removing the dead skin, corn or hardened skin from the skin.

In order to prevent the dead skin, corn or hardened skin particles while rubbing them with the dead skin removing apparatus 100 from contaminating the surroundings, most of the dead skin, corn or hardened skin particles removed by the dead skin removing blade 114 are collected in the through hole 112 during the removing work, and part of them is collected in the collection chamber 124 formed between the body 110 and the protrusion ring 122.

The protrusion ring 122 is concentrically formed at the outer side of the dead skin removing apparatus 114, so both the dead skin removing blade 114 and the protrusion ring 122 can be concurrently contacted with the skin, and then the dead skin removing work is performed. In this state, the dead skin removing bade 114 can protrude by a certain depth, which makes wider a skin contacting area, so it is possible to remove in safe dead skin, corn or hardened skin without damaging skin.

When the dead skin removing apparatus 100 is no used, the dead skin removing blade 114 of the body 110 is covered by the cap 130, so the dead skin removing apparatus can be stored in safe and moved.

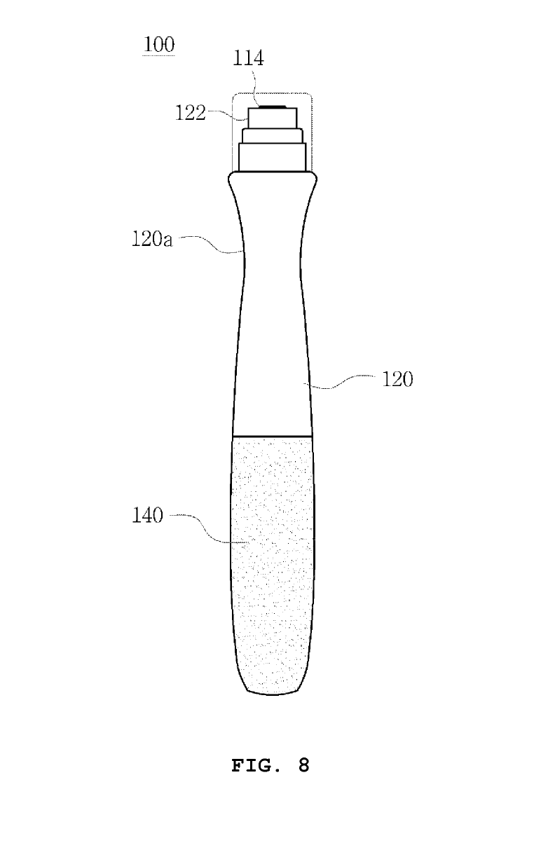

FIG. 8 is a view illustrating a dead skin removing apparatus according to another embodiment of the present invention.

As is best seen therein, a polishing ember 140 is provided at an outer surface of the grasping member 120 so a user can polish and remove dead skin depending on situation.

At this time, the polishing member 140 can be formed on the whole outer surfaces of the grasping member 120 or can be selectively formed at one of the center, front side and backside about the center of the grasping member 120.

The grasping member 120 has a concave groove 120a which allows the user to easily gasp.

Since the operations of the dead skin removing apparatus of the present invention are same as the earlier descriptions, the descriptions on the operation will be omitted.

In this embodiment, dead skin can be removed using the polishing member 140 formed at the outer surface of the grasping member 120.

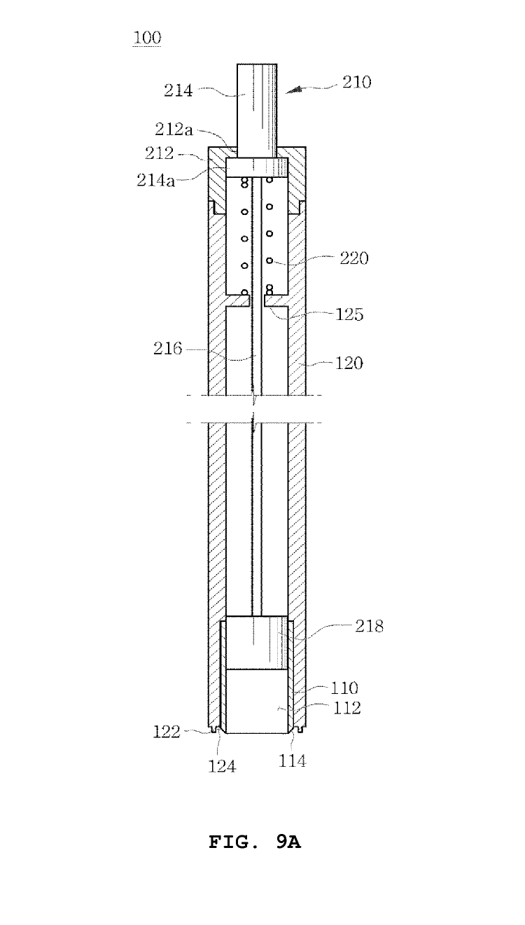

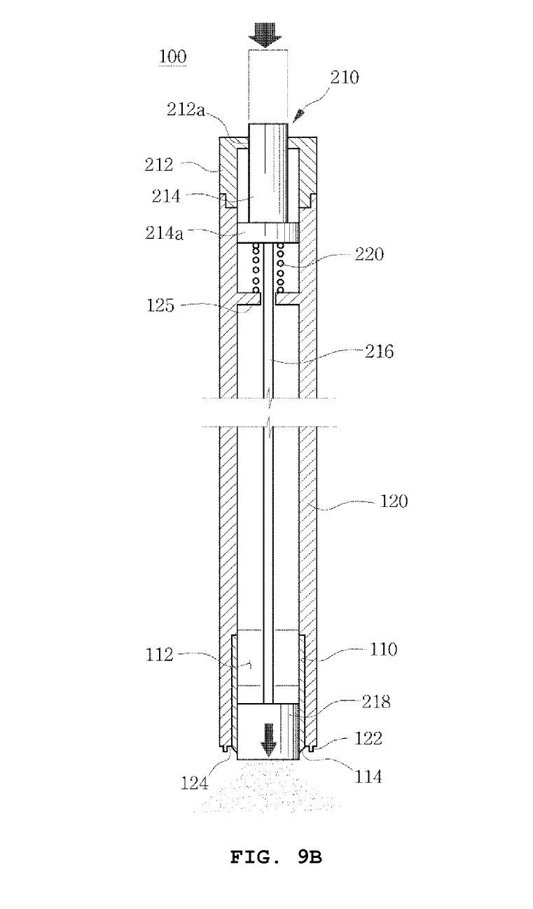

FIG. 9A is a view illustrating a dead skin removing apparatus according to another embodiment of the present invention.

As is best seen therein, there is further provided a dead skin particle removing part 210 which serves to remove the dead skin particles collected in the through hole 112 of the body 110, which dead skin particles are produced while the user removes dead skin, corn or hardened skin using the dead skin blade 114 formed at the body 110.

Here, the dead skin particle removing part 210 has a support ring 125 the center of which passes through the inner surface of the grasping member 120.

A cover member 212 is fixedly engaged to the other side of the graphing member 120 to which the body 110 is engaged. The cover member 212 has a slide hole 212a which is stair-shaped at the center.

A button 214 is fixedly engaged to the outer side of the slide hole 212a of the cover member 212 and has an engaging shoulder 214a supported by the inner surface of one side of the cover member 212.

A connection rod 216 is positioned at an inner side of the grasping member 120. One side of the connection rod 216 is fixedly engaged to the button 214.

At the other side of the connection rod 216 fixedly engaged to the button 214 is provided a removing member 218 which serves to discharge outside dead skin particles collected in the through hole 112 of the body 110.

There is provided an elastic member 220 which is inserted into the connection rod 216 and is installed between the support ring 125 and the button 214 for thereby elastically supporting the button 214.

Since the operations of the thusly constituted dead skin removing apparatus of the present invention are same as the earlier descriptions, the description thereon will be omitted.

As is best seen in FIG. 9B, in this embodiment, the dead skin particles which are produced when removing dead skin, corn or hardened skin using the dead skin removing apparatus 100 penetrate into the through hole 112 of the body 110 and are collected at the front side of the removing member 218.

The user presses the button 214 in a state that the dead skin particles are collected in the through hole 112 of the body 110, the button 214 presses the elastic member 220 into the inner side of the cover member 212.

As the button 214 slides, the removing member 218 connected to the connection rod 216 moves along the inner surface of the through hole 112 of the body 110, so the dead skin particles collected at the front side of the removing member 218 are discharged to the outside of the body 110.

In this state, when the force being applied to the button 214 is removed, the button 214 returns to its original position by the elastic force of the elastic member 220, and the removing member 218 connected to the button 214 through the connection rod 216 moves backward, so the returning procedure is finished.

The dead skin removing apparatus 100 can be kept clean all the time because the dead skin particles collected in the through hole 112 of the body 110 can be easily removed after the dead skin removing apparatus 100 is used.

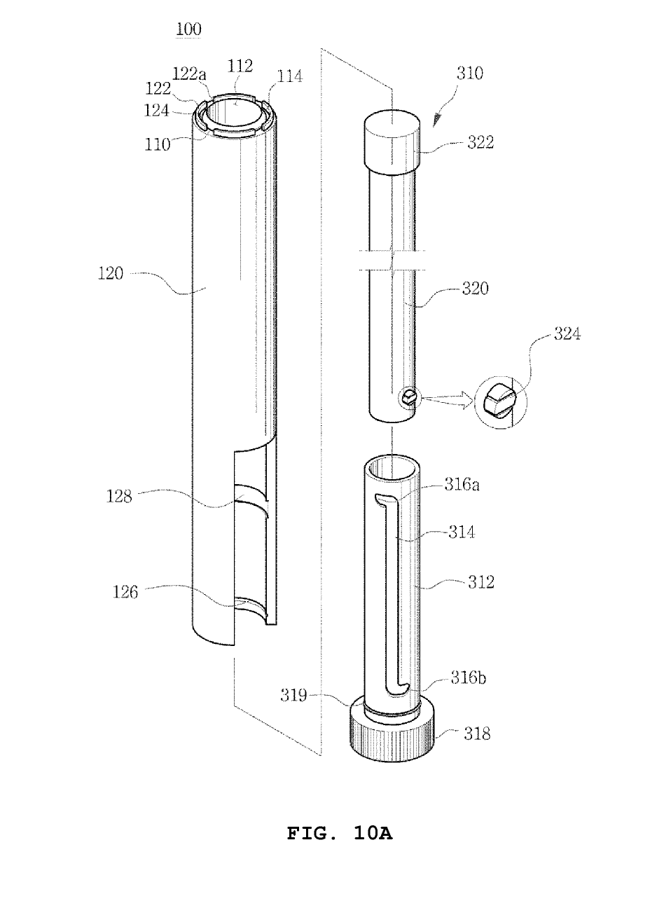

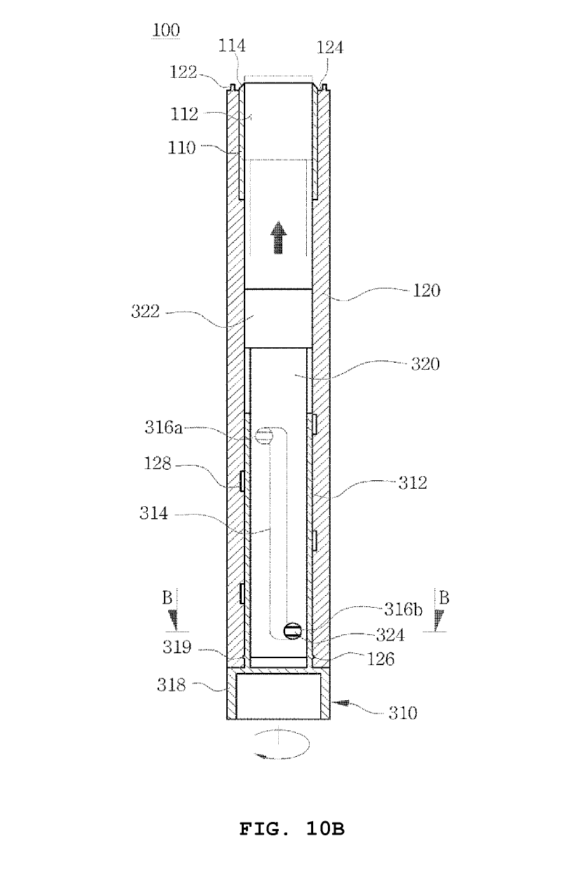

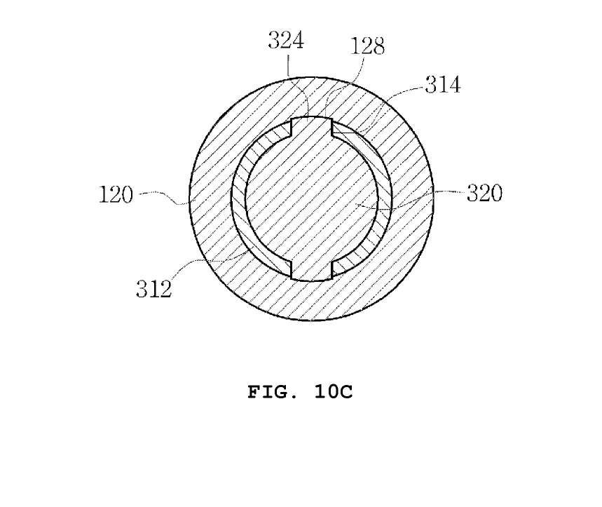

FIGS. 10A and 10B are views illustrating a dead skin removing apparatus according to another embodiment of the present invention, and FIG. 10C is a cross sectional view taken along line B-B of FIG. 10B.

As is best seen therein, the dead skin particle removing part 310 has an insertion groove 126 formed at an inner surface of the grasping ember 120, and the insertion groove 126 is formed at the inner surface of the reverse direction of the direction where the body 110 is connected. A screw groove 128 is formed in the direction where the insertion groove 116 is formed.

In addition, there is provided a rotational body 312 which is inserted into the inner side of the grasping member 120 and is inserted into the other side to which the body 110 is fixedly engaged. One side of the rotational body 312 is stair-shaped and protrudes in the direction of the outside of the grasping member 120.

The rotational body 312 is formed in a tubular shape, one side of which is inserted into the inner side of the grasping member 120. The rotational body 312 has an ascending and descending guide groove 312 with engaging grooves 316a and 316b at both ends in crossing shapes, and a handle 318 protrudes at the lower end in the direction of the outside of the grasping member 120.

The rotational body 312 comprises an insertion groove 319 which us formed at an inner surface of the grasping member 120 and is engaged to the insertion groove 126.

Therefore the insertion protrusion 319 of the rotational body 312 keeps being engaged to the insertion groove 126 of the grasping member 120, so the rotational body 312 does not separate from the grasping member 120 while performing a rotational operation in the engaged state.

There is provided an ascending and descending holder 320 which is positioned at the inner side of the grasping member 120 and ascends and descends in a state that it is inserted at the inner side of the rotational body 312.

One side of the ascending and descending holder 320 is stair-shaped for thereby forming an ascending and descending member 322 positioned at the through hole 112 of the body 110, and the other side of the same is inserted into the inner side of the rotational body 312. At the outer side of the ascending and descending holder 320 positioned at the inner side of the rotational body 312 is formed a guide protrusion 324 which is engaged to the screw groove 126 of the grasping member 120 after it passes through the ascending and descending guide groove 314 of the rotational body 312.

The guide protrusion 324 is provided in one pair. The screw groove 128 formed at the inner surface of the grasping member 120 through which the guide protrusion 324 passes and the ascending and descending guide groove 314 formed at the rotational body 312 are provided in pairs.

Since the operations of the above described dead skin removing apparatus of the present invention are same as the earlier descriptions, the descriptions thereon will be omitted.

In this embodiment, part of the dead skin, corn or hardened skin particles which are produced when removing them penetrates into the through hole 112 of the body 110 and is collected at the front side of the ascending and descending holder 320.

The user grasps the rotational body 312 in a state that dead skin particles are collected in the through hole 112 of the body 110 and rotates the rotational body 312 in one direction. The ascending and descending holder 320 engaged to the screw groove 128 of the grasping member 120 through the ascending and descending guide groove 314 formed at the rotational body 312 moves in the direction that the dead skin removing blade 114 is formed, along the screw groove 128 with the aid of the rotation of the rotational body 312.

At this time, the ascending and descending holder 320 inserted in the ascending and descending guide groove 314 rotates the guide protrusion 322. As the guide protrusion 322 rotates, the guide protrusion 322 moves in one direction along the screw groove 128 formed at the inner side of the grasping member 120.

In this state, when the ascending and descending holder 320 moves and reaches the end portion of the ascending and descending guide groove 314 of the rotational body 32, it is mounted at the engaging groove 316a formed at one side of the ascending and descending guide groove 314, so the rotational body 312 no longer rotates, and the ascending and descending holder 320 keeps the stop state.

As the ascending and descending holder 320 moves by the rotations of the rotational body 312, the dead skin particles collected in the through hole 112 of the body 110 are discharged to the outside by the ascending and descending member 322.

When the rotational body 312 is rotated in the reverse direction after the dead skin particles collected in the through hole 112 of the body 110 are discharged outside, the ascending and descending holder 320 moves in the reverse direction of the direction where the dead skin particles are discharged outside.

As the ascending and descending holder 320 moves in the reverse direction, it is mounted at the engaging groove 316b formed at the opposite side of the ascending and descending groove 314 of the rotational body 312, so the ascending and descending holder 320 no longer moves and keeps being stopped.

Therefore, the dead skin removing apparatus 100 of the present invention can be kept clean all the time because it is possible to easily remove dead skin particles collected in the through hole 112 of the body 110 after the dead skin removing apparatus 100 is used.

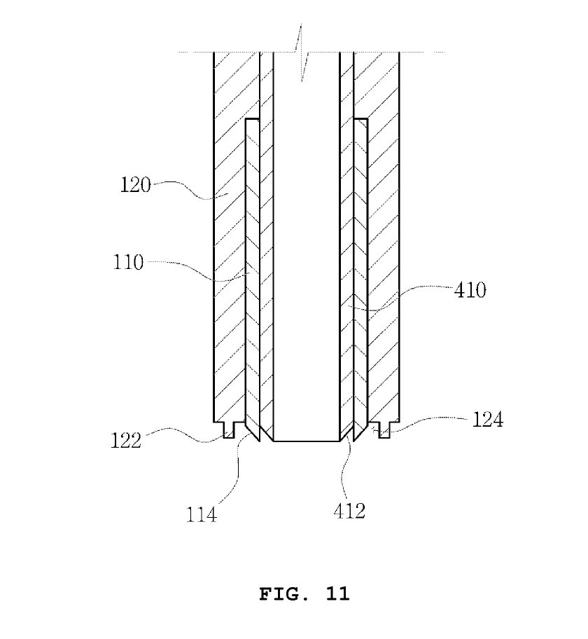

FIG. 11 is a view illustrating a dead skin removing apparatus according to another embodiment of the present invention.

As best seen therein, there is provided an assistant body 410 which is installed at the through hole 112 of the body 110 and has an assistant body with an assistant dead skin removing blade 4 412 while corresponding to the dead skin removing blade 114 of the body 110.

Since the operations of the above described dead skin removing apparatus are same as the earlier descriptions, the descriptions thereof will be omitted.

In this embodiment, the blade functioning to remove dead skin is formed in a dual structure which makes it possible to more efficiently remove dead skin, corn or hardened skin.

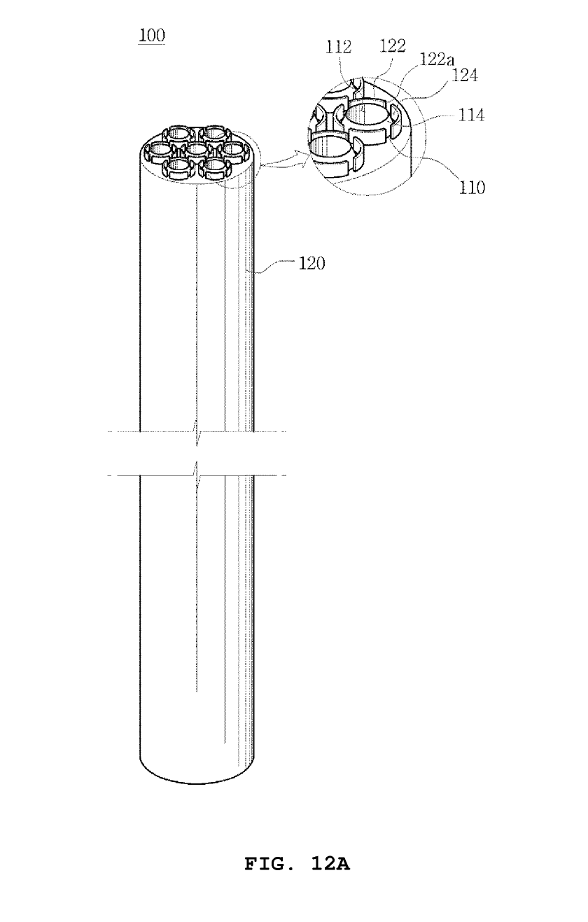

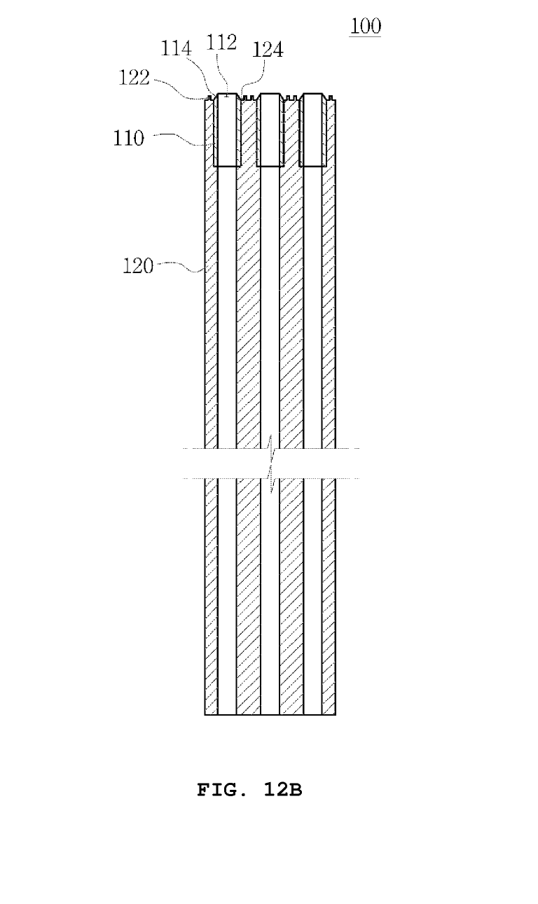

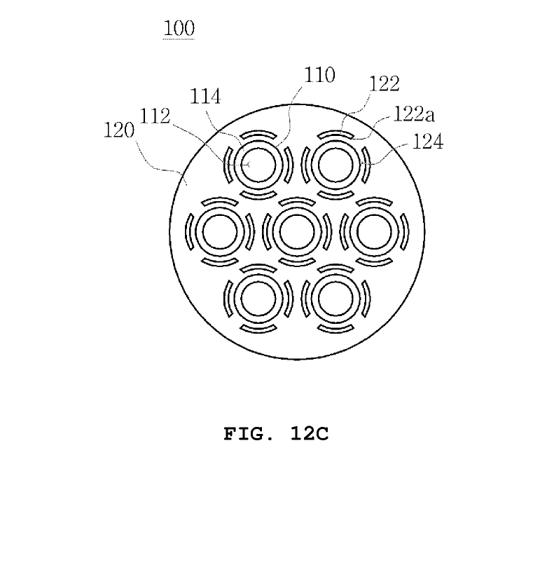

FIGS. 12A to 12C are views illustrating a dead skin removing apparatus according to another embodiment of the present invention.

As is best seen therein, a plurality of bodies 110 are fixedly engaged to the grasping member 120 with one side of each of them being outwardly protruded.

More specifically, a plurality of bodies 110 are fixedly engaged to the grasping member 120 so that a dead skin removing blade 114 formed at the body 110 can be outwardly protruded in the direction of the outer side of the grasping member 120.

At this time, at one side of the grasping member 120 formed at the body 110 are provided a plurality of protrusion rings 122 concentrically to the dead skin removing blade 114.

Since the operations of the above described dead skin removing apparatus are same as the earlier descriptions, the descriptions thereof will be omitted.

In this embodiment, a plurality of bodies are provided in group using the grasping member 120 for thereby forming one dead skin removing apparatus 100, so it is possible to more efficiently remove dead skin, corn or hardened skin in wider area.

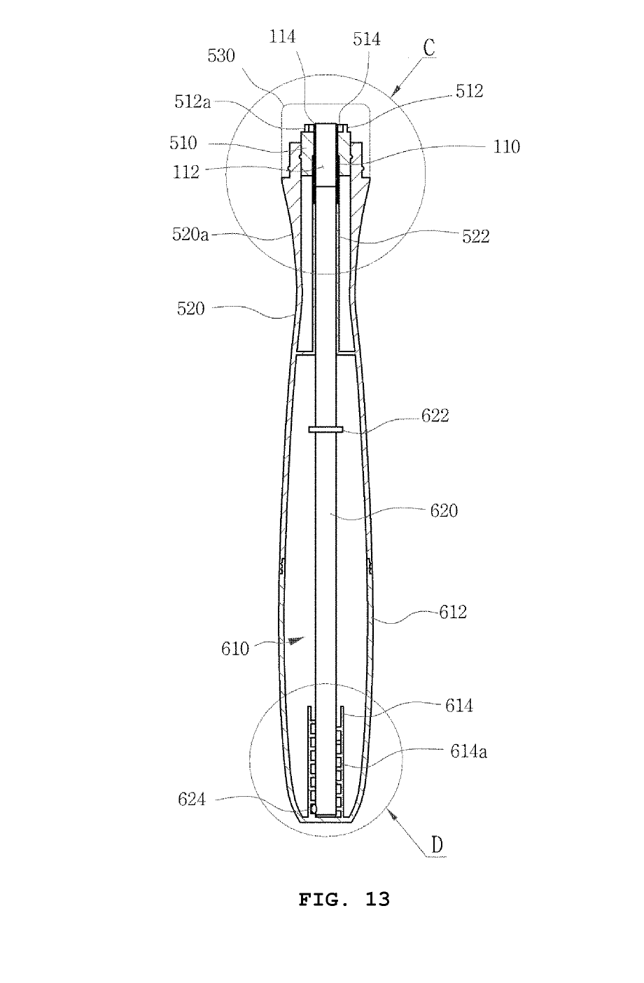

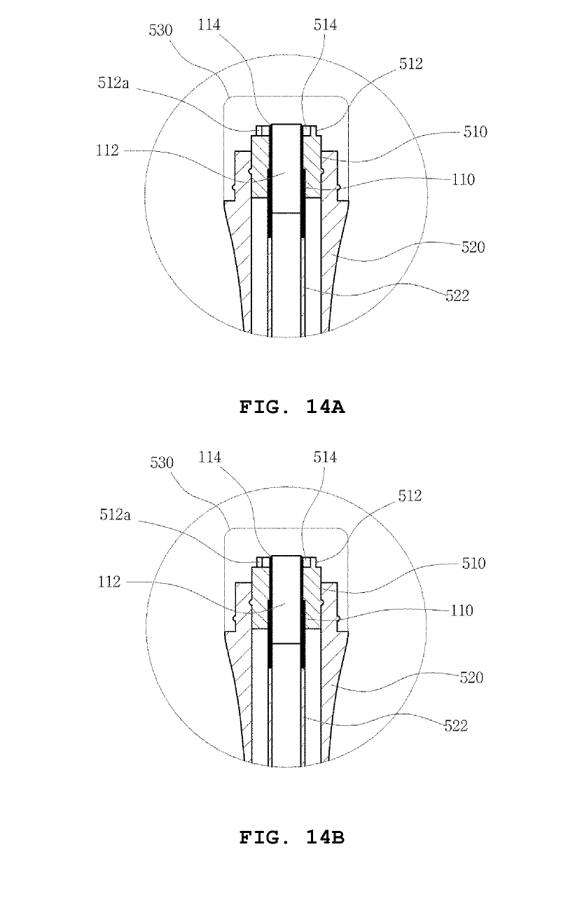

FIG. 13 is a view illustrating a dead skin removing apparatus according to still further another embodiment of the present invention. FIG. 14A is an enlarged view of a portion “C” of FIG. 13. FIG. 14B is an enlarged view of a portion “D” of FIG. 13.

As is best seen therein, the dead skin removing apparatus according to another embodiment of the present invention has features in that there is provided a body 110 in which a through hole 112 is formed at an inner side, and which has a dead skin removing blade 114 which is provided at one side in a longitudinal direction at its one side.

At this time, the body 110 is formed in a tubular shape and has a dead skin removing blade 114 at one outer side in a slanted angle.

In addition, there is provided a support member 510 at an outer side of the body 110, to which support member 510 is engaged the body 110.

Here, the support member 510 is fixedly engaged in order for the outer side of the dead skin removing blade 114 of the body 110 to be covered. The support member 510 is fixedly engaged to the outer side of the body 110 in such a way that the end portion of the dead skin removing blade 114 can protrude a certain length.

A ring-shaped protrusion ring 512 is formed at one side of the support member 510 so that a collection chamber 514 can be formed between itself and the outer surface of the body 110 at a spaced distance from the outer side of the body 110 in the direction of one side of the support member 510, more specifically, in the direction where the dead skin removing blade 114 of the body 110 is formed.

There is provided a grasping member 520 to which the support member 510 is fixedly engaged and which is fixedly engaged so that one side with the protrusion ring 512 can be outwardly protruded.

At this time, the grasping member 520 has a concave groove 520a which is formed in a concave shape in order for a user to easily grasp.

A tubular guide member 522 is integrally formed at the inner side of the grasping member 520. The guide member 522 has the same inner diameter as the body 110.

At this time, the guide member 522 is formed at the inner side of the grasping member 520. The body 110 comes into close contact with one side of the guide member 522. The guide member 520 extends in the reverse direction of the direction when the body 110 closely contacts.

There is provided a cap 530 which is fixedly engaged to the grasping member 520 and serves to cover the dead skin removing blade 114 of the body 110.

There is provided a dead skin particle removing part 610 which is configured to remove dead skin particles collected in the through hole 112 of the body 110 which are produced when removing dead skin, corn or hardened skin using the dead skin removing blade 114 formed at the body 110.

Here, the dead skin removing part 610 is rotatable-engaged at the bottom of the grasping member 520, and a tubular engaging member 614 is formed at the same axis as the guide member 522 at the inner side. To the inner surface of the engaging member 614 is fixedly engaged a rotational body 612 with a screw groove 614a.

At this time, the engaging member 614 is formed at an inner side in the reverse direction of the direction when it is fixedly engaged to the grasping member 520.

There is provided a slide rod 620 at the inner sides of the grasping member 520 and the rotational body 612. One side of the slide rod 620 is positioned at an inner side of the through hole 112 of the body 110, and the other side of the same is engaged to an inner side of the engaging member 614 formed in the rotational body 612.

At one side of the slide rod 620 is formed an engaging protrusion 622 which is selectively supported by the guide member 522. At one side of the same is provided a ball 624 which is engaged to the screw groove 614a formed at the inner side of the engaging member 614.

Since the operations of the dead skin removing apparatus of the above-described embodiment of the present invention are same as the earlier descriptions, the descriptions on the operation will be omitted.

In this embodiment, part of the dead skin particles produced when removing dead skin, corn or hardened skin using the dead skin removing apparatus 100 may penetrate into the through hole 112 of the body 100.

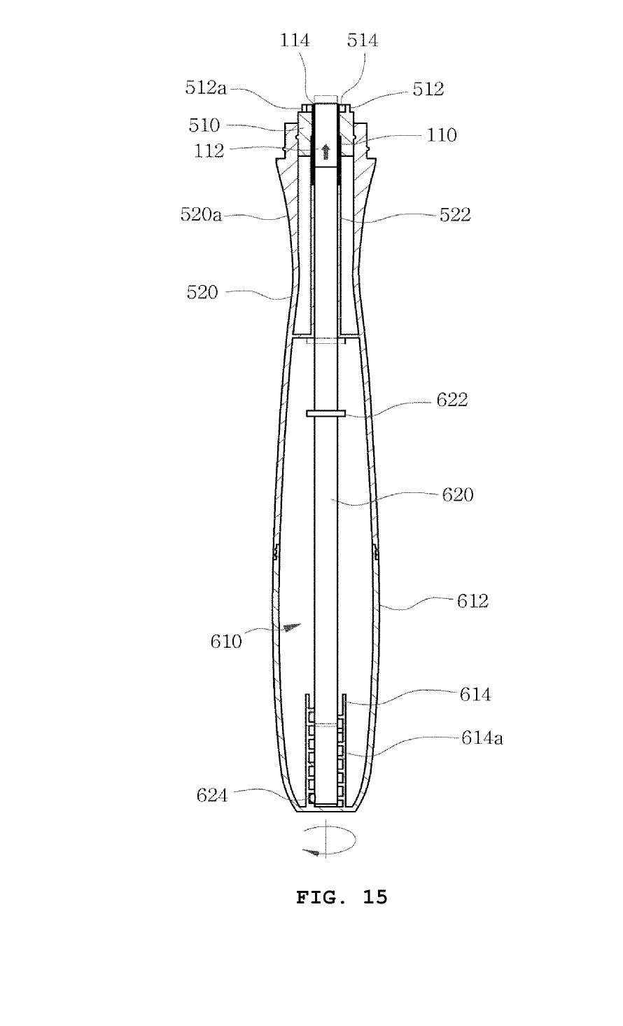

When a user rotates in one direction the rotational body 612, as shown in FIG. 15, in a state that the dead skin particles are collected in the through hole 112 of the body 110, the ball 624 moves along the screw groove 614a formed at the inner surface of the engaging member 614 of the rotational body 612, and the slide rod 620 moves in the direction where the dead skin removing blade 114 is formed.

At this time, the slide rod 620 keeps being engaged to the screw groove 614a of the engaging member 614 in a state that the ball 624 is fixed to the slide rod 620, so the ball 620 can move along the screw groove 614a during the rotation of the rotational body 612, whereby the slide rod 620 moves forward and backward.

In this state, as the engaging protrusion 622 formed at the slide rod 620 comes into close contact with the guide member 522 after the slide rod 620 moves, the rotational body 612 no longer rotate, so the movement of the slide rod 620 is stopped.

Therefore, the slide rod 620 moves by the rotation of the rotational body 612, and the dead skin particles collected in the through hole 112 of the body 110 are pushed outwardly by the slide rod 620 and are discharged.

When the rotational body 612 rotates in the reverse direction after the dead skin particles collected in the through hole 112 of the body 110 are discharged outside, the ball 624 moves along the screw groove 614a in the reverse direction of the direction when the slide rod 620 moves forwards.

As the ball 624 moves along the screw groove 614a in the reverse direction, the slide rod 620 moves backward, and one side protruded toward the outer side of the body 110 is inserted into the through hole 112 of the body 110.

The dead skin removing apparatus 100 can be kept clean all the time since the dead skin particles collected in the through hole 112 of the body 110 after the dead skin removing apparatus 100 can be easily removed.

As the present invention may be embodied in several forms without departing from the spirit or essential characteristics thereof, it should also be understood that the above-described examples are not limited by any of the details of the foregoing description, unless otherwise specified, but rather should be construed broadly within its spirit and scope as defined in the appended claims, and therefore all changes and modifications that fall within the meets and bounds of the claims, or equivalences of such meets and bounds are therefore intended to be embraced by the appended claims.

### INDUSTRIAL APPLICABILITY

The present invention relates an apparatus for removing dead skin, and more particularly, to an apparatus for removing dead skin which can safely remove hardened skin or dead skin generated on the soles, heels, and palms without damaging normal skin, and simultaneously easily remove dead skin particles remaining within the apparatus for removing dead skin.

1. An apparatus for removing dead skin, comprising:
a tubular body having a dead skin removing blade at its one side; and
a grasping member which is formed at an outer portion of the body.

2. An apparatus for removing dead skin, comprising:
a tubular body having a dead skin removing blade at its one side;
a support member formed at an outer side of the body; and
a grasping member to which the support member is fixedly engaged.

3. The apparatus of claim 1, wherein a protrusion ring is formed concentrically to a dead skin removing blade of the body.

4. The apparatus of claim 3, wherein the protrusion ring is formed at a grasping member.

5. The apparatus of claim 3, wherein the protrusion ring is formed at a support member.

6. The apparatus of claim 3, wherein the protrusion ring has a plurality of cut-away grooves.

7. The apparatus of claim 6, further comprising a cap which is fixedly engaged to an outer surface of the protrusion ring.

8. The apparatus of claim 1, wherein a polishing member is formed at an outer surface of the grasping member.

9. The apparatus of claim 1, wherein a dead skin particle removing part is provided at an inner side of the body.

10. The apparatus of claim 9, wherein the dead skin removing part comprise:
a support ring which is provided at an inner surface of the grasping member;
a cover member which serves to cover the reverse direction of the direction where the body is engaged and has a slide hole in a stair-shape at its one side;
a button which is engaged to the slide hole of the cover member;
a connection rod which is provided at an inner side of the grasping member and one side of which is engaged to the button;
a removing member which is fixedly engaged to the other side to which the button is fixedly engaged; and
an elastic member which is engaged to the connection rod and is positioned between the support ring and the button.

11. The apparatus of claim 9, wherein the dead skin particle removing part comprises a rotational body in which a screw groove is formed at an inner surface of the grasping member, and an insertion groove is formed at an inner surface of the other side to which the body is fixedly engaged, and a handle is inserted into the inner side of the grasping member and has an insertion protrusion engaged to the insertion groove and is stair-shaped and protrudes toward an outer side of the grasping member, and an ascending and descending guide groove with crossing engaging grooves is formed at both ends in a longitudinal direction, and the rotational body has an ascending and descending member inserted into a through hole of the body as one side protrudes outwardly and is formed in a stair-shape, and at a lateral side is provided an ascending and descending holder having a guide protrusion which passes through the ascending and descending guide groove and is engaged to the screw groove of the grasping member.

12. The apparatus of claim 9, wherein the dead skin particle removing part comprises a slide rod in which a tubular guide member is provided at an inner side of the grasping member, and a rotational body is rotatable-engaged to the bottom of the grasping member, and a tubular engaging member is formed at one inner side of the rotational body and is positioned on the same axis as the guide member, and a screw groove is formed at an inner surface of the engaging member, and one end portion of the slide rod passes through the guide member and is positioned in the through hole of the body, and the other side is engaged to the engaging member, and a ball is provided at the engaging part of the engaging member.

13. The apparatus of claim 12, wherein the slide rod comprises an engaging protrusion.

14. The apparatus claim 1, wherein the dead skin removing blade comprises an assistant dead skin removing blade at its outer side.

15. The apparatus of claim 1, wherein the bodies are provided in multiple numbers.

16. The apparatus of claim 2, wherein a protrusion ring is formed concentrically to a dead skin removing blade of the body.

17. The apparatus of claim 2, wherein a polishing member is formed at an outer surface of the grasping member.

18. The apparatus of claim 2, wherein a dead skin particle removing part is provided at an inner side of the body.

19. The apparatus claim 2, wherein the dead skin removing blade comprises an assistant dead skin removing blade at its outer side.

20. The apparatus of claim 2, wherein the bodies are provided in multiple numbers.
